(19) United States
(12) Patent Application Publication
(10) Pub. No.: US 2010/0160887 A1
(43) Pub. Date: Jun. 24, 2010

(54) Lily pad panty

(76) Inventors: Ruby Lee Brown, Woodbridge, VA (US); Harry Ulric Hodgson, Alexandria, VA (US)

Correspondence Address: Ruby L. Brown, 13771 Musket Ct., Woodbridge, VA, 22193, US

(21) Appl. No.: 12/584,654

(22) Filed: Sep. 10, 2009

Related U.S. Application Data
(60) Provisional application No. 61/191,500, filed on Sep. 10, 2008.

(51) Int. Cl. A61F 13/45; A61F 13/15; A41D 7/00

(52) U.S. Cl. 604/385.11; 604/396; 2/67

(57) ABSTRACT

The present invention relates to a panty for ladies that can be worn during menstruation. These panties are for protection against leakage while swimming, sunbathing, or just relaxing in the hot tub. Thus the name Lily Pad Panty. The present invention Lily Pad Panty is made of a unique absorbent material. They are disposable. Sides can be torn away for ease of removal.

This Application claims the benefit of the Provisional Application 61/191,500

### BACKGROUND OR PROBLEM

Some women are uncomfortable wearing tampons underneath their bathing suits. However, they have no choice in the matter if they are menstruating. Currently, no product exists that gives an alternative to this dilemma. Others who feel awkward wearing tampons, such as young ladies who start menstruating early in life, choose not to wear tampons because they are uncomfortable.

### SOLUTION

The Lily Pad Panty allows a female to swim during menstruation. The product can serve as a replacement for tampons when menstruating women wear bathing suits.

### DETAILED DESCRIPTION OF INVENTION

The Lily Pad Panty is waterproof underwear that resembles a pair of bikini bottoms. The garment is comprised with a unique absorbent material that does not leak, allowing a menstruating woman to wear the panty in a pool or other body of water during menstruation. The panty is constructed with elastic, tear away sides, and the panty can expand if a woman is moving actively. When the garment is finished being worn, it tears off easily and can be thrown away. A prototype of the device exists and has been worn in a pool by menstruating women. Exact specifications of the device may vary when it is manufactured.

### DETAILED DESCRIPTIONS OF DRAWINGS ATTACHED

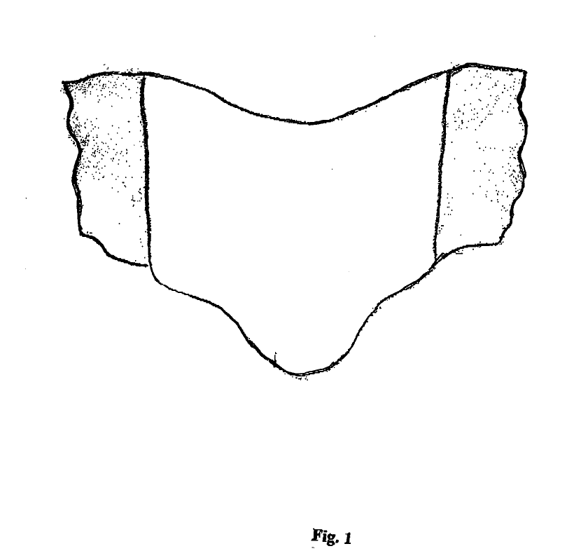

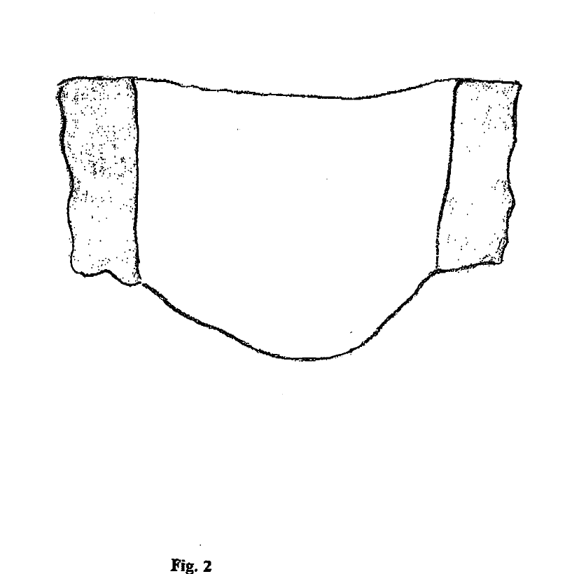

FIG. 1 shows a diagram explaining the different components of the garments.

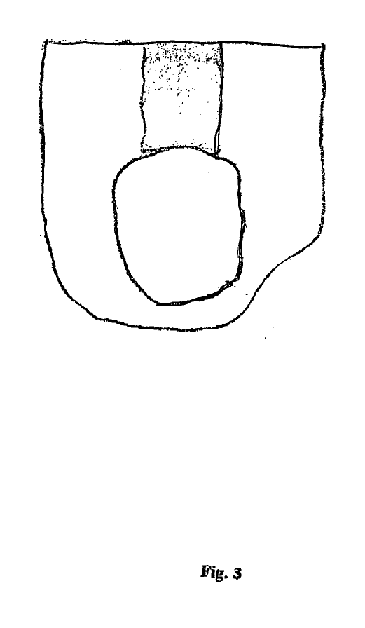

FIG. 2 shows a back view of the Lily Pad Panty.

FIG. 3 shows a side view of the panty.

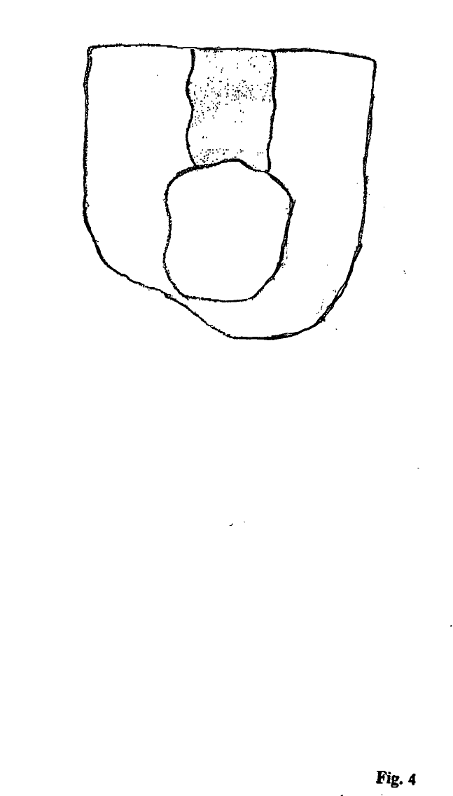

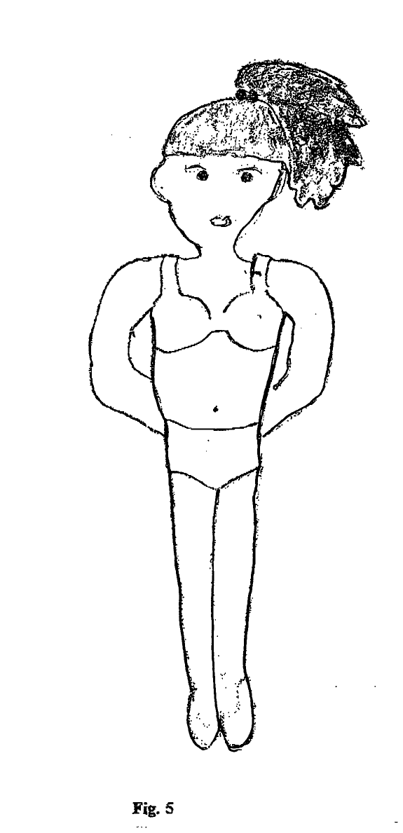

FIG. 4 shows a model comfortably wearing the garment.

Although the concepts of the present disclosure have been illustrated and described in detail in the drawings and descriptions, they are for example only and not restricted in character. Any changes and modification that come within the spirit the disclosure are to be protected by the following claims:

1. This is a waterproof panty in the shape of bikini bottoms.

2. This can be worn in the water while menstruating.

3. The panty will not leak as per claim 1.

4. It is disposable and easy to remove.

5. It does have tear away sides for ease of removal as per claim 4.

6. Its shape is in the form of a bikini bottom as per claim 1.
